(19) United States
(12) Patent Application Publication
(10) Pub. No.: US 2004/0028610 A1
(43) Pub. Date: Feb. 12, 2004

(54) Method of increasing the sensitivity of mammography

(76) Inventor: Stephen E. Zimberg, Plantation, FL (US)

Correspondence Address: Pamela A Docherty, Calfee Halter & Griswold, 1400 McDonald Investment Center, 800 Superior Avenue, Cleveland, OH, 44114, US

(21) Appl. No.: 10/381,824

(22) PCT Filed: Jul. 31, 2001
(86) PCT No.: PCT/US01/23995

(51) Int. Cl. A61K 49/00; A61K 31/57

(52) U.S. Cl. 424/9.1; 514/177

(57) ABSTRACT

Methods of improving detection of cancerous lesions in the breast through breast examination are provided. Such methods comprise administering to a patient in need of a breast examination, an androgen, preferably testosterone or a derivative thereof, in an amount sufficient to reduce the palpable breast tissue density or radiographic breast tissue density of the patient. Methods of treating conditions associated with dense breasts in a patient are also provided. Such methods comprise administering, a biologically effective amount of an androgen to a patient in need of the same. In accordance with the present invention, it has been found that administration of testosterone to pre- and post-menopausal women with fibrous or dense breasts results in a decrease in palpable breast density, radiographic density, and the number or size of fibrous breast lesions in these women.

### BACKGROUND OF THE INVENTION

The present invention relates to pharmaceutical preparations and methods for increasing sensitivity of mammography and other breast tissue examination techniques, thereby improving the detection of breast cancer.

Breast cancer affects a large segment of the female population over the age of 40, and is the leading cause of cancer death in women. A woman has a 1 in 8 chance of developing the disease during her lifetime. As with most cancers, early detection is key to survival. The standard clinical methods for detecting breast cancer are self-examination, manual clinical examination, radiography of the breast (known as mammography), and biopsy.

Breast self examination is manual examination of a woman's breast tissue by the woman herself. Clinical breast examinations are routinely performed by gynecologists or other primary care physicians. During clinical breast examination, clinicians generally examine the breast manually to determine whether any foreign structures are encountered. The effectiveness of manual clinical breast examination depends on several factors including the individual physician's proficiency, the duration of the examination, and the weight and age of the patient. Manual examinations are ineffective for identifying non-palpable cancerous growths and are not always appropriate or sensitive enough for detecting breast lesions. In particular, manual examinations may not be effective if the patient's breasts are especially dense or contain large amounts of fibrocystic tissue.

Mammography can be used to identify cancerous growths even before a mass is palpable. Mammography employs specially designed mammography equipment and involves imaging structures that have small differences in x-ray radiation absorption characteristics. During this procedure, breast tissue is optimally positioned so that as much tissue as possible can be imaged to produce films known as mammograms. The radiologist then evaluates the image shown on the mammogram for presence of suspicious tissue. Suspicious findings include any abnormalities in the breast tissue. Such abnormalities may be either malignant or benign. The ability of the radiologist to accurately detect the presence of a potentially malignant lesion depends on the quality of the image. In approximately 10 - 30% of women who are ultimately found to have breast cancer, the results of mammography are either negative or indeterminate due to poor mammographic image quality. Overall, up to 75% of mammograms may be deemed indeterminate. In such cases, successful detection relies on a tissue biopsy. However, 70% of such biopsies turn out to be benign. Unnecessary biopsies are costly in terms of physical and emotional discomfort to the patient.

Improvements in mammographic equipment have overcome many of the technical problems that previously limited the ability of radiologists to assess fine details in mammograms. Despite these advances, certain features of breast tissue, such as tissue density, obscure the presence of lesions. Women with dense breasts have been shown to have a four to six-fold increased risk of developing breast cancer.

The term “dense breast” refers to the condition of the breast wherein the parenchyma is very fibrous and dense. This condition makes manual self examination difficult since dense breasts are often not palpable. This condition also makes mammography difficult since the dense breast is not easily compressed during mammography. Moreover, it is also difficult for the radiologist to accurately read the mammogram of a patient with dense breasts.

Radiographic breast density relates to the fact that x rays permeate different types of breast tissue differently. Each type of breast tissue reacts differently to x-rays. Fatty breast tissue is relatively translucent, allowing x-rays to pass through providing dark areas on a mammogram. Epithelial tissue and stromal tissue, however, block x-rays and appear as white on film. Since dense tissue and tumors both look white on film, breast lesions are not easily discernible in areas which contain substantial amounts of epithelial and stromal tissue.

Presently, approximately 30% of women have dense breast tissue. Thus, despite the great advances made in breast cancer detection and successful treatment, a large segment of the patient population remains at risk for missed diagnosis, or exposure to unnecessary biopsies.

Accordingly, it is desirable to have new methods and preparations for increasing the sensitivity of the methods used to detect breast cancer.

### SUMMARY OF THE INVENTION

The present invention provides a method of improving detection of cancerous lesions in the breast through breast examination, particularly through mammography. Such method comprises administering to a patient in need of a breast examination, an androgen, preferably testosterone or a derivative thereof, in an amount sufficient to reduce the palpable breast tissue density or radiographic breast tissue density of the patient. In accordance with the present invention, it has been determined that administration of testosterone in female patients with dense breast tissue results in significantly improved visualization of breast tissue in mammographic procedures.

The present invention also provides a method of treating conditions associated with dense breasts in a patient. These conditions including palpable breast density, radiographic breast density, and fibrocystic properties. Such method comprises administering to a patient in need of the same, a biologically effective amount of an androgen. In accordance with the present invention, it has been found that administration of testosterone to pre- and post-menopausal women with fibrous or dense breasts results in a decrease in palpable breast density, radiographic density, and the number or size of fibrous breast lesions in these women. It was also found that administration of testosterone to these women eliminated symptoms secondary to cyclic causes, such as a sense of breast fullness or heaviness.

The present invention also provides preparations of testosterone and other androgens that are suitable for reducing radiographic density and palpable density of breast tissue.

### DETAILED DESCRIPTION OF THE INVENTION

The present disclosure provides novel methods and preparations to assist clinicians in diagnosing breast cancer more accurately and reducing the incidence of indeterminate mammograms. Such methods comprise administering a pharmaceutical preparation comprising a biologically effective amount of an androgen, particularly testosterone and its derivatives, to a patient in need of the same. Advantageously, such method reduces the need for biopsies and may permit earlier detection of lesions in the population of patients with dense breasts. Advantageously, the preparations and methods of this invention provide additional benefits for patients suffering from disorders related to dense or polycystic breasts.

In accordance with the present method, it has been determined that administration of testosterone to female patients alters breast tissue. In patients presenting with dense breast tissue and attendant mastalgia, androgen therapy was employed and resulted in significantly improved palpability of breast tissue during manual examination, and marked reduction in mammographic opacity and overall density. Androgen therapy also reduced the number and/or size of fibrocystic lesions in the breasts of female patients and the symptoms associated with cyclic causes. Such symptoms include a sense of breast heaviness or fullness.

### 1. PREPARATIONS AND FORMULATIONS OF ANDROGENS

The pharmaceutical preparations of the present invention comprise a biologically effective amount of one or more androgens, preferably testosterone or a derivative thereof, and preferably, a relatively inert carrier. Many such carriers are routinely used and can be identified by reference to pharmaceutical texts. The acceptable carrier is a physiologically acceptable diluent or adjuvant. The term physiologically acceptable means a non-toxic material that does not interfere with the effectiveness of the androgen. The characteristics of the carrier will depend on the route of administration and particular compound or combination of compounds in the composition. Preparation of such formulations is within the level of skill in the art. The preparation may further contain other agents that either enhance the activity of the androgen or complement its activity. The preparation may further comprise fillers, salts, buffers, stabilizers, solubilizers, and other materials well known in the art.

Suitable androgens include, but are not limited to, those listed in Table 1 that may be adapted for use in pharmaceutical preparations in accordance with the present invention. Preferably, the preparation comprises testosterone or a derivative thereof. The term “testosterone derivative” as used herein encompasses 5α-dihydrotestosterone (DHT), and Δ⁴—androstenedione, and other testosterone derivatives known in the art. Preferably the preparation comprises testosterone in one of its salt forms, such as testosterone propionate, testosterone cypionate, or testosterone enanthate. A highly preferred parenteral formulation is Testosterone Cypionate 200 mg/ml dissolved in Cottonseed Oil and preserved with Benzyl Benzoate USP and Benzyl Alcohol NF.

TABLE 1

| | Proprietary/ | |
|---|---|---|
| Generic Name | Trade Name | Dosage Form |
| Testosterone | Testoject-50 ™ | Aqueous susp. for IM use |
| | Testex ™ | Oily solution for IM use |
| Testosterone Enanthate | Delatestyl | Oily solution for IM use |
| Testosterone Cypionate | Depo- | Oily solution for IM use |
| | Testosterone | |
| Nandrolone Decanoate | Deca-Durabolin | Oily solution for IM use |
| Nandrolone | Durabolin | Oily solution for IM use |
| | Phenpropionate | |
| Danazol | Danocrine | Capsules |
| Fluoxymesterone | Halotensin | Tablets |
| Methandrostemolone | Dianabol | Tablets |
| Methyltestosterone | Metandren, | Tablets and Capsules |
| | Oreton Methyl | Daily. Buccal Tablets |
| Oxandrolone | Anavar | Tablets |
| Oxymetholone | Anadrol-50 | Tablets |
| Stanozolol | Winstrol | Tablets |
| Testolactone | Teslac | Tablets |

Various formulations for delivery of the androgen preparation include oral tablets or capsules, vaginal or rectal suppositories, single or multiple injections, aeorosol preparations and topical formulations. Delivery of pharmaceutical agents by oral tablets, suppositories and injection is well known in the art.

Aerosol or nebulized administration, can be achieved through the nasal or buccal route, or by inhalation into the lungs. Inhalant preparations include the active ingredient, i.e., the androgen, preferably supplied in finely dispersed form, together with a surfactant and a propellant. Both the active agent and the surfactant are nontoxic, and preferably soluble in the propellant. Liquefied propellants are typically gases at ambient conditions, and are condensed under pressure.

Topical preparations include creams, gels, and transdermal patches. Another formulation for administration of an androgenic preparation is topical application of a volatile liquid composition to the skin of a patient. The composition will usually contain an alcohol such as ethanol or isopropanol. Other agents or carriers may be present in such topical formulations.

### 3. DOSAGE AND ADMINISTRATION OF THE ANDROGENIC PREPARATION

The androgen preparation is administered to the patient, preferably a female patient, in a biologically effective amount. As used herein, the term “biologically effective amount” means the total amount of androgen that is sufficient to show a meaningful benefit, i.e., a reduction in palpable breast tissue density as determined by physician and patient physical examination, or a reduction in mammographic or radiographic density as determined on mammographic images. The BI-RADS system, which is standard scheme of categorization of breast tissue based upon its radiographic properties, may be used to determine the extent of radiographic breast tissue density. The BI-RADS system of the American Society of Radiologists is a four group categorization scheme in which breast tissue is classed as either entirely fat, having scattered fibroglandular tissue, heterogeneously dense, or extremely dense. (See Breast Imaging Reporting And Data System, American College of Radiology Report, BI-RADS (May 19, 1993):1-22.) Alternatively, a computer program that calculates the percentage density using objective criteria (See White (2000) J. Natl. Cancer Inst.; 92:6, 443) is used to compare the radiographic breast tissue density before treatment and after treatment.

The effective amount of the androgen preparation may vary depending upon the route of administration. It follows that the androgen preparation may be more effective when administered by some routes, but less effective when administered by other routes, and that dosages may be adjusted accordingly.

The amount of androgen in the formulation will depend upon the nature and extent of breast cell density or extent of fibrocystic changes and on the mammographic density. Ultimately, the attending physician will decide the amount of androgen with which to treat each individual subject. Initially, the attending physician will administer lower doses of androgen and observe the patient's response. Larger doses of androgen may be administered until the optimal effect is obtained for the patient, and at that point, the dosage is not increased further. It is contemplated that the various androgen preparations used to practice the method of the present invention should contain from about 50 mg to about 300 mg per monthly dose, preferably from about 100 mg to about 200 mg per monthly dose, and most preferably from about 150 mg to about 200 mg per monthly dose.

Although a single dose of androgen may be sufficient to reduce breast tissue density, it is expected that multiple single doses or slow release forms of the androgenic preparation will be preferred. It is highly preferable that the patient self-administer the androgenic preparation according to the dosage schedule prescribed by the attending physician or other responsible clinician. Optionally, the androgenic preparation may be administered in the clinical office setting by a health care professional.

Highly preferred androgen preparations are slow release formulations, such as suspensions in oil for injection, or compounds in a patch matrix and carrier, that would allow slow release of between about 1 mg and about 7 mg equivalents per day. The advantage of such slow release formulations is the convenience and cost savings of avoiding clinical office visits. A further advantage of such formulations is the greater efficacy observed with such topical formulations in comparison to the less efficacious oral delivery. Yet another advantage of such formulations is improved patient compliance due to the relative ease and reduced frequency of self-applications.

Preferably, the androgenic preparation is administered from about 90 to about 30 days prior to mammography or alternative forms of breast examination, such as for example physical examination, thermograms, MRI or CT scans or newer imaging technology.

### 3. POPULATION

The preparations and methods of this invention are suitable for use with any patient population in which there is a need for breast cancer screening. The preferred target population is female patients, and most preferably those female patients who have radiographically dense or palpably dense breasts. Optionally, such patients may also present with other non-malignant breast disorders such as breast tenderness or mastalgia, and impalpable or fibrocystic breasts. Another preferred target population are those individuals with a family history or genetic risk for developing breast cancer.

### 4. EXAMPLES

The following examples are for purposes of illustration only.

### Example 1

Treatment with Parenteral Formulations of Androgens

Patients in need of breast cancer examinations were provided parenteral administration of androgen preparations in the clinical setting. Lakeview Pharmacy, Inc. prepared the compounded equivalent of Depo-Testosterone (Testosterone Cypionate 200 mg/ml dissolved in Cottonseed Oil and preserved with Benzyl Benzoate USP and Benzyl Alcohol NF.) in a multi-use vial. The androgen preparation was administered in a dose of about 150 mg to about 200 mg per month for the treatment of fibrocystic breast disorders. The treating physician delivered the preparation intramuscularly in a single dose. The preparation was given to approximately 40 pre and postmenopausal women ranging in age from 24 to 80 years of age. The patients were then scheduled for a mammogram at least one month but not more than three months after commencement of administration of the androgen preparation.

In advance of the mammographic procedure, patients were instructed to not wear deodorant (the crystals cause difficulty in reading the films). During the mammographic procedure, the breast being imaged was placed on a flat plate and a compression plate was used to compress the breast in the superior-inferior direction. A Picker Digital Mammogram was used to take the frontal and side views of the breast tissue bilaterally. Minimums of two views were taken per breast. The films were then interpreted by a radiologist with experience in interpreting mammograms and graded with the BI-RADS system.

Mammographic images from patients who received androgen treatment revealed appreciable reduction in radiographic breast density, and improved breast palpability by manual examination. These effects on radiographic and palpable density were observed in patients who received only one monthly treatment approximately one month prior to the examination. More significant effects were observed in patients who received two months of androgen treatment prior to examination. Mammographic changes in density were noted in all patients but were most significant in those patients with dense breasts. In addition, some lesions requiring 6-month follow up had disappeared by the second mammogram after sustained treatment for 6 or more months. The observed effect of the androgen treatment on radiographic density was not influenced by the menstrual cycle nor did the medication alter the menstrual cycle.

### Example 2

Treatment with Topical Formulations of Androgens

Patients in need of breast cancer examinations were provided topical formulations for administration of androgen preparations in the outpatient setting. Lakeview Pharmacy, Inc made up the compounded equivalent of Depo-Testosterone (150 mg of testosterone cypionate mixed in PCA cream) with an application of 6 inches squeezed from a multi-use tube of the mixture and rubbed into the skin on a daily basis. The androgen preparation was administered as a total monthly dose from about 150 mg to about 200 mg. The formulation was given to approximately 15 pre and postmenopausal women ranging in age from 24 to 80 years of age. The patient was then scheduled for a mammogram at least one month but not more than two months after administration of the androgen preparation. Mammography was performed as described in Example 1.

Mammographic images from patients who received androgen treatment revealed appreciable reduction in radiographic breast density, and improved breast palpability by manual examination. These effects on radiographic and palpable density were observed in patients who received only one monthly treatment approximately one month prior to the examination. More significant effects were observed in patients who received two months of androgen treatment prior to examination. Mammographic changes in density were noted in all patients but were most significant in those patients with dense breasts. In addition, some lesions requiring 6-month follow up had disappeared by the second mammogram after sustained treatment for 6 or more months. The observed effect of the androgen treatment on radiographic density was not influenced by the menstrual cycle nor did the medication alter the menstrual cycle.

### Example 3

Treatment with Transdermal Patch Comprising Androgens

Patients in need of breast cancer examinations are provided transdermal topical formulations for administration of androgen preparations in the outpatient setting. Such formulations comprise a transdermal patch delivering doses of androgen equivalent of from about 0.5 mg to about 10 mg per day, and most preferably about 1.5 mg to about 7 mg per day (100 mg to 200 mg per month). Dosage is in the form of a weekly patch, self administered by the patient for a period from about 2 weeks to about 2 months prior to mammography, and packaged as 5 patches per month pack and a 10-patch pack. The patient is then scheduled for a mammogram at least one month but not more than two months after commencement of administration of the androgen preparation. Mammography is performed as described in Example 1.

Response to androgen treatment is evaluated by manual examination and mammography. Affect on radiographic breast density is quantified according the BI-RADS system.

### Example 4

Treatment with Inhalants Comprising Androgens

Patients in need of breast cancer examinations are provided inhalant formulations for administration of androgen preparations in the outpatient setting. Such formulations comprise a delivery device containing a mixture of the androgen preparation, preferably testosterone or a testosterone derivative, an appropriate carrier agent, a surfactant and a propellant, for delivering doses of androgen equivalent of from about 0.5 mg to about 10 mg per day, and most preferably about 1.5 mg to about 7 mg per day (100 mg to 200 mg per month). Dosage is in the form of a multi-dose unit, self administered by the patient for a period from about 2 weeks to about 2 months prior to mammography. The patient is scheduled for a mammogram at least one month but not more than two months after commencement of administration of the androgen preparation. Mammography is performed as described in Example 1.

Response to androgen treatment is evaluated by manual examination and mammography. Affect on radiographic breast density is quantified according the BI-RADS system.

While the preparations and methods of this invention have been described in their preferred embodiments, it will be evident to those of skill in the art that many variations may be introduced to the formulations of the preparations, and the steps and sequence of steps embodied in the methods, consistent with the scope and the underlying concept of the invention. Moreover, it will be evident to one of skill in the art that substitutions of pharmaceutical and preparations and vehicles described in the preferred embodiments are deemed to be within the scope and spirit of the invention where such substitutions are either chemically or physically related to the preparations or vehicles, or would result in the same or similar results described herein.

What is claimed is:

1. A method of improving breast tissue visualization in mammography of a patient, such method comprising administering to the patient a biologically effective amount of an androgen.

2. The method of claim 1 wherein said androgen is testosterone or a testosterone derivative.

3. The method of claim 1 wherein said testosterone or testosterone derivative is administered to the patient in a dose from about 100 mg to about 200 mg.

4. The method of claim 1 wherein the patient is a female patient with radiographically dense breasts.

5. The method of claim 4 wherein at least 5% of the breast tissue of the patient is radiographically dense.

6. The method of claim 5 wherein at least 25% of the breast tissue of the patient is radiographically dense.

7. The method of claim 1 wherein the androgen is administered to the patient within three months of the mammography.

8. The method of claim 6 wherein the androgen is administered to the patient multiple times prior to the mammography.

9. The method of claim 1 wherein the androgen is administered to the patient at regular dose intervals for at least one month prior to said breast cancer examination.

10. The method of claim 9 wherein said regular dose interval is either daily, weekly, or monthly.

11. The method of claim 1 wherein the patient has impalpable or fibrocystic breasts.

12. The method of claim 1 wherein the patient has a family history or genetic risk for developing breast cancer.
